SEQUENCE LISTING (8 sequences)

SEQ ID NO: 1 - DNA, 20 bp, Artificial Sequence
  Description of Artificial Sequence Synthetic primer
gggaaggtgaaggtcggagt

SEQ ID NO: 2 - DNA, 25 bp, Artificial Sequence
  Description of Artificial Sequence Synthetic primer
gacgaaaattgagaccatttcacct

SEQ ID NO: 3 - DNA, 15 bp, Artificial Sequence
  Description of Artificial Sequence Synthetic primer
tgtccagaattgcag

SEQ ID NO: 4 - DNA, 23 bp, Artificial Sequence
  Description of Artificial Sequence Synthetic primer
tggatggataagaattcgcccgt

SEQ ID NO: 5 - DNA, 21 bp, Artificial Sequence
  Description of Artificial Sequence Synthetic primer
atgctggtgctgagttcgtgg

SEQ ID NO: 6 - DNA, 22 bp, Artificial Sequence
  Description of Artificial Sequence Synthetic primer
cctcggttttcccagtagtaga

SEQ ID NO: 7 - DNA, 24 bp, Artificial Sequence
  Description of Artificial Sequence Synthetic primer
atctctgcttcgttgggctcatct

SEQ ID NO: 8 - DNA, 24 bp, Artificial Sequence
  Description of Artificial Sequence Synthetic primer
gtagggtagtgtcggtttcggtaa

(19) United States
(12) Patent Application Publication
(10) Pub. No.: US 2011/0306570 A1
(43) Pub. Date: Dec. 15, 2011

(54) Na/K-Atpase Expression as an Indicator for the Treatment of Cancer

(75) Inventors: Zi-Jian Xie, Saline, MI (US); Jiang Tian, Perrysburg, OH (US)

(73) Assignee: UNIVERSITY OF TOLEDO, Toledo, OH (US)

(21) Appl. No.: 13/126,022

(22) PCT Filed: Oct. 28, 2009
(86) PCT No.: PCT/US09/62317

§ 371 Date: Jun. 10, 2011

Related U.S. Application Data
(60) Provisional application No. 61/109,386, filed on Oct. 29, 2008.

(51) Int. Cl. A61K 31/7048; C12N 5/09; C40B 30/04; C40B 30/06; G01N 33/566; C12Q 1/68; A61K 31/585; A61P 35/00

(52) U.S. Cl. 514/26; 514/171; 435/375; 506/10; 435/7.23; 435/6.12; 506/9

(57) ABSTRACT

Methods for regulating the expression of Na/K-ATPase and uses thereof, including uses in the diagnosis/prognosis and treatment of cancer, are disclosed.

### CROSS-REFERENCE TO RELATED APPLICATIONS

This application claims the benefit of U.S. Provisional Application No. 61/109,386 filed Oct. 29, 2008, the disclosure of which is incorporated herein by reference.

### STATEMENT REGARDING FEDERALLY SPONSORED RESEARCH

This invention was made with Government support and the Government has rights in this invention under the National Institutes of Health Grants HL-36573 and HL-67963, awarded by the National Institute of General Medical Sciences, United States Public Health Service, Department of Health and Human Services.

### REFERENCE TO SEQUENCE LISTING SUBMITTED VIA EFS-WEB

This application is being filed electronically via the USPTO EFS-WEB server, as authorized and set forth in MPEP§1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows: 420_{—}50446_SEQ_LIST_D2009-09.txt, created on Oct. 23, 2009, and is 2,092 bytes in size.

### TECHNICAL FIELD AND INDUSTRIAL APPLICABILITY OF THE INVENTION

The present invention is based, in part, on the discovery that ouabain-induced changes in the expression of Na/K-ATPase dictate cell growth regulation. Further, ouabain-induced activation of PI3-K/Akt/mTOR pathway is a key to ouabain-induced up-regulation of the Na/K-ATPase. Also, the inhibition of PI3-K/Akt/mTOR pathway by rapamycin (or drugs similar to rapamycin) can change the ouabain response from growth stimulation to growth inhibition.

### BACKGROUND OF THE INVENTION

The Na/K-ATPase, a member of P-type ATPase family, was discovered as an energy transducing ion pump. It transports Na+ and K+ across the cell membrane and maintains ion homeostasis in animal cells.

Recent studies indicate that the Na/K-ATPase is also an important receptor that can confer ligand binding into the activation of protein kinase cascades. Specifically, the Na/K-ATPase interacts with Src, which produces at least two important cellular regulations. First, it keeps Src in an inactive state. Thus, the Na/K-ATPase serves as a native negative Src regulator. Second, this interaction forms a functional receptor complex for cardiotonic steroids, a group of well-characterized ligands of Na/K-ATPase. Cardiotonic steroids (CTS) include cardenolides (e.g., ouabain) and bufadienolides (e.g., MBG-marinobufagenin). Although CTS are known cardiac drugs, some of them have now been identified as endogenous steroid hormones. Binding of CTS to the receptor complex activates the Na/K-ATPase-associated Src. Subsequently, the activated Src transactivates other tyrosin kinases and together they recruit and further phosphorylate multiple membrane and soluble proteins, which results in the activation of protein kinase cascades and the generation of second messages. Ultimately, this chain of signaling events would alter cellular functions and cell growth in a cell-specific manner. For instance, the inventors and others have demonstrated that ouabain-induced activation of ERK and PI3-K/Akt/mTOR pathways are responsible for cell growth stimulation in transformed cell lines, primary cultures as well as in vivo.

It has also been recognized for long time that CTS inhibit cell growth in many cancer cells. Of particular significance are studies that indicate the benefit effects of CTS therapy in women with breast cancer. Consistently, recent in vitro and in vivo studies have identified several new CTS compounds that exhibit anti-cancer activities. Oleandrin, for example, is in clinical trials in the United States as an anti-cancer remedy for human cancers. Although ouabain inhibits the pumping function of the Na/K-ATPase, it is important to note that the growth inhibitory effect of ouabain can occur at the doses that neither cause significant changes in intracellular Na+ and K+ nor affect cell viability. Rather, like its effect on cell growth stimulation, ouabain induces cell growth inhibition through activation of protein kinases and generations of second messengers. For example, a recent report showed that these non-toxic concentrations of ouabain stimulated Src, resulting in the activation of the EGFR/ERK pathway and induction of the expression of cell cycle inhibitor p21^{cip} (an inhibitor of cyclin-dependent protein kinases) and cell growth arrest. Thus, it becomes important to understand the molecular mechanisms that govern different fates of cells in response to CTS stimulation, so that a method can e developed to either inhibit or stimulate cell growth.

Currently, the Na/K-ATPase is the only known receptor of CTS. Prior studies have demonstrated that CTS induce endocytosis of the Na/K-ATPase and regulate its cellular expression via the receptor-mediated signal transduction. Because the Na/K-ATPase has both pumping and signaling functions, the inventors herein now believe that changes in cellular Na/K-ATPase amount could have significant consequences on cell growth.

The inventors herein have now shown the role of cellular Na/K-ATPase in ouabain-induced cell growth regulation and discovered means to modulate cellular amount of Na/K-ATPase. Considering the above-mentioned concerns, it is clear that there remains a need in the art for a method of targeting the newly discovered Na/K-ATPase/Src receptor complex to allow CTS, such as ouabain, digoxin, and digitoxin, to either inhibit or stimulate cell growth.

Such a method would also contribute to the development of increasingly more effective therapeutic, diagnostic, or prophylactic agents having fewer side effects.

According to the present invention, just such a method is provided.

### SUMMARY OF THE INVENTION

In a first broad aspect, there is provided herein a method to determine whether a cancer patient may receive a therapeutic benefit from a cardiotonic steroid (CTS) treatment, comprising: determining whether cancer cells in the patient have lost the ability to increase cellular synthesis of Na/K-ATPase, wherein a loss of Na/K-ATPase synthesis indicates that the patient may benefit from the CTS treatment.

In certain embodiments, the method is useful for the diagnosis and/or prognosis of a cancer patient intending to receive digitalis treatment.

In certain embodiments, the method comprises determining whether a ouabain-induced Na/K-ATPase expression change in the cancer cell is directly related with the ouabain-induced cell growth regulation.

In certain embodiments, the method includes detecting PI3-Kinase activation in the cancer cell which affects ouabain-induced Na/K-ATPase up-regulation in the cancer cell.

In certain embodiments, the method includes measuring the Na/K-ATPase expression change in the cells.

In certain embodiments, the method includes measuring using a Western blot or 3H-ouabain binding assay.

In another broad aspect, there is provided herein a method for determining whether a cell is susceptible to a cardiotonic steroid (CTS) treatment, comprising: determining whether the cell maintains the ability to replete reduced-Na/K-ATPase in the cell.

In certain embodiments, the method includes determining whether the cell lacks a mechanism to compensate for ouabain-induced Na/K ATPase loss. In certain embodiments, the cell is a cancer cell.

In another broad aspect, there is provided herein a method of modulating cellular synthesis of Na/K-ATPase, comprising administering an effective amount of a Na/K-ATPase inhibitor, such as a cardiotonic steroid CTS, so as to modulate the apparent biological activity of Na/K-ATPase.

In another broad aspect, there is provided herein a method for treating a patient, comprising administering to the patient an effective amount of a Na/K-ATPase inhibitor, either alone or in combination with at least one: the target of mTOR inhibitor and/or phosphoinisitide 3-kinase (PI3-K) inhibitor.

In another broad aspect, there is provided herein a pharmaceutical formulation comprised of a Na/K-ATPase inhibitor in combination with at least one: the target of mTOR inhibitor and/or phosphoinisitide 3-kinase (PI3-K) inhibitor, formulated in a pharmaceutically acceptable excipient and suitable for use in human patients. In certain embodiments, the Na/K-ATPase inhibitor is a cardiotonic steroid (CTS).

In another broad aspect, there is provided herein a kit for treating a patient having cancer, comprising a Na/K-ATPase inhibitor, either alone or in combination with a composition which reduces expression of Na/K-ATPase

In another broad aspect, there is provided herein a method of identifying cancer patients less likely to respond to cardiotonic steroid (CTS) therapy, the method comprising:

obtaining a biological sample from a cancer patient wherein the biological sample comprises cancer cells; and

determining Na/K-ATPase levels or activity, wherein a patient having a change in NF Na/K-ATPase levels or activity, as compared to the Na/K-ATPase levels or activity found in a normal healthy subject indicates that the patient is less likely to respond to CTS therapy.

In another broad aspect, there is provided herein a method of diagnosing a cancer or providing a prognosis for patient having a cancer that has altered expression of molecular signaling pathways triggered by ouabain, the method comprising:

contacting a sample of the cancer with an antibody that specifically binds to protein that is part of a molecular signaling pathway triggered by ouabain, wherein the molecular signaling pathway is a functional or activated PI3-K/Akt/mTOR pathway; and

determining whether or not expression of the protein is altered in the sample, thereby diagnosing or providing the prognosis for the cancer.

In another broad aspect, there is provided herein a method of identifying a compound that inhibits a cancer that has an altered molecular signaling pathway triggered by ouabain, the method comprising the steps of:

contacting a cell expressing a polypeptide member of the molecular signaling pathways triggered by ouabain with a compound, wherein the molecular signaling pathway is a functional or activated PI3-K/Akt/mTOR pathway; and

determining the effect of the compound on the polypeptide; thereby identifying a compound that inhibits the cancer.

In another broad aspect, there is provided herein a method of identifying a compound that inhibits a therapy resistant cancer, the method comprising:

contacting a cell expressing a polypeptide member of a molecular signaling pathways triggered by ouabain with a compound, wherein the molecular signaling pathway is a functional or activated PI3-K/Akt/mTOR pathway; and

determining the effect of the compound on the polypeptide; thereby identifying a compound that inhibits the therapy resistant cancer.

In another broad aspect, there is provided herein a method of treating or inhibiting a cancer in a subject that that has an altered molecular signaling pathway triggered by ouabain comprising:

administering to the subject a therapeutically effective amount of one or more inhibitors that modulates a polypeptide member of a molecular signaling pathway triggered by ouabain, wherein the molecular signaling pathway is a functional or activated PI3-K/Akt/mTOR pathway.

In another broad aspect, there is provided herein a method of treating or inhibiting a therapy resistant cancer in a subject comprising:

administering to the subject a therapeutically effective amount of one or more modulators of a molecular signaling pathway triggered by ouabain, wherein the molecular signaling pathway is a functional or activated PI3-K/Akt/mTOR pathway.

In certain embodiments, the therapy resistant cancer has an altered expression of the molecular signaling pathway triggered by ouabain, and the therapy-resistant cancer was diagnosed by:

contacting a sample of the cancer with an antibody that specifically binds to protein that is part of a molecular signaling pathway triggered by ouabain; and

determining whether or not expression of the protein is altered in the sample, thereby diagnosing or providing the prognosis for the cancer.

In certain embodiments, tone or more modulators are administered concurrently with another cancer therapy.

In certain embodiments, the method comprises multiple determinations of a pattern of expression levels, at different points in time, thereby allowing the monitoring of the development of the cancer in the subject.

In certain embodiments, the method comprises an estimation of the likelihood of success of a given mode of treatment for the cancer in the subject.

In another broad aspect, there is provided herein a method for regulating cell growth, comprising causing ouabain- or other cardiotonic (CTS)-induced changes in the expression of Na/K-ATPase in the cell.

In another broad aspect, there is provided herein a method for regulating cell growth, comprising:

modulating the expression of Na/K-ATPase in a cell by contacting the cell with a CTS to induce activation of the PI3-K/Akt/mTOR pathway in the cell with an amount sufficient for ouabain-induced up-regulation of the Na/K-ATPase.

In another broad aspect, there is provided herein a method for assessing the effectiveness of CTS therapy in a patient receiving such CTS therapy, comprising monitoring expression of Na/K-ATPase in such patient.

In certain embodiments, the patient suffers from cancer. In certain embodiments, the method includes administering an effective amount of rapamycin along with the CTS therapy in the treatment of cancer. In certain embodiments, the cancer comprises breast cancer and the CTS therapy comprises administering about 1 to about 50 nM ouabain, digoxin or digitoxin.

In another broad aspect, there is provided herein a method to replete the plasma membrane pool of functional Na/K-ATPase in a cell in need thereof, comprising affecting ouabain-induced up-regulation of ouabain-sensitive α1 isoform of Na/K-ATPase (α1) in the cell.

In certain embodiments, the method includes administering at least one CTS sufficient to decrease plasma membrane Na/K-ATPase and cause ouabain-induced growth inhibition in cancer cells.

In another broad aspect, there is provided herein a method for sensitizing a cell to ouabain-induced cell growth inhibition, comprising:

administering an effective amount of rapamycin sufficient to block ouabain-induced cell growth in the cell.

In another broad aspect, there is provided herein a method for increasing the expression of the expression of Na/K-ATPase in a cell in need thereof via a PI3-K/Akt/mTOR-dependent translational mechanism, comprising administering ouabain or other CTS at a low concentration.

In another broad aspect, there is provided herein a method for blocking ouabain-induced up-regulation of Na/K-ATPase in a cell in need thereof, comprising inhibiting the PI3-K/Akt/mTOR pathway in the cell.

In certain embodiments, the method includes administering an effective amount of one or more of a PI3-K inhibitor or mTOR inhibitor.

In certain embodiments, the method includes administering rapamycin or an analog thereof.

In another broad aspect, there is provided herein a method for restoring cell growth, comprising administering an effective amount of ouabain sufficient to inhibit expression of p21^{cip} regulatory protein.

In another broad aspect, there is provided herein a pharmaceutical formulation comprising ouabain or other CTS in combination with an inhibitor of the PI3-K/AKt/mTOR pathway, formulated in a pharmaceutically acceptable excipient and suitable for use in humans to treat cancer.

In another broad aspect, there is provided herein a kit for treating a patient having cancer, comprising ouabain or other CTS in combination with an inhibitor of PI3-K/AKt/mTOR pathway, each formulated in premeasured doses for administration to the patient.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1C: Effects of ouabain on cell growth in different cells.

FIG. 1A: LLC-PK1 cells, BT20 cells, and DU145 cells were subcultured in 96-well plates (10,000 cells/well) and serum starved. After 24 h treatment with different concentrations of ouabain, MTT reagent was added. Cells were solublized by adding 100 μl/well detergent provided by ATCC after 90 min of incubation and OD570 was then measured.

FIG. 1B: The above three cell lines were subcultured in 12-well plates (50,000 cells/well) and serum starved. After ouabain treatment, 3 well of control and ouabain-treated cells were trypsinized and counted at indicated time points.

FIG. 1C: LLC-PK1, BT20, and DU145 cells were serum-starved overnight and treated with ouabain for 24 h. The cell lysates were collected and probed for p21cip using Western blot. * p<0.05, ** p<0.01.

FIGS. 2A-2C: Long-term ouabain treatment differentially regulates Na/K-ATPase expression and membrane abundance in LLC-PK1, BT20 and DU145 cells:

FIG. 2A: Long-term ouabain treatment regulated Na/K-ATPase α1 expression measured by Western blot.

FIG. 2B: LLC-PK1 and BT20 cells were treated with 10 nM ouabain for 24 h and the cell lysates were assayed for Na/K-ATPase 131 using Western blot.

FIG. 2C: LLC-PK1, BT20, and DU145 cells were pre-treated with ouabain for 72 h. The cells were washed and 3H-ouabain were performed as described herein. * p<0.05, ** p<0.01

FIGS. 3A and 3B: Reduction of Na/K-ATPase by siRNA sensitized cells to digitalis-induced cell growth inhibition:

FIG. 3A: The control cells (P11) and Na/K-ATPase knock-down cells (A4-11 and PY17) were treated with different concentrations of ouabain for 16 h and the cell growth was measured by MTT assay.

FIG. 3B: The Na/K-ATPase knock-down cells (PY17) were treated with different digitalis compounds and the cell growth was measured by MTT assay.

FIGS. 4A-4C: Reduction of Na/K-ATPase by siRNA regulates cell growth and cell cycle through induction of p21cip:

FIG. 4A: Cell growth curve of P11, PY17 and AAC-19 cells were measured by subculturing the cells in 12-well plates (50,000 cells/well) and 3 wells of each cell type were trypsinized and counted at the indicated time points, data was normalized to the seeded cell number.

FIG. 4B: P11, PY 17 and AAC-19 cells were lysed in RIPA buffer and analyzed by

Western blot using anti-p21cip antibody. A representative Western blot showed the p21cip expression in the three cell lines.

FIG. 4C: Cell cycle was measured as described herein. Briefly, P11, PY17 and AAC-19 were cultured in 10-cm Petri dishes for 24 h. Cells were trypsinized and resuspended in citrate buffer and followed by PI staining. The cell cycle was then measured by flow cytometry.

FIG. 5: Ouabain does not affect Na/K-ATPase α1 mRNA levels. LLC-PK1, BT20, and DU145 cells were treated with 10 nM ouabain for 24h. Total RNA was extracted and applied for RT-PCR to probe Na/K-ATPase α1 as described in the Experimental Procedures section.

FIGS. 6A-6C: Ouabain accelerates Na/K-ATPase degradation through endocytosis:

FIG. 6A: LLC-PK1 cells, BT20 cells, and DU145 cells were treated with 10 nM ouabain for 6 h, and fixed with cold methanol. Na/K-ATPase al was immunostained and visualized using Leica confocal microscope. The red arrows indicate the endocytosed vesicles containing Na/K-ATPase α1.

FIG. 6B: LLC-PK1 cells were serum starved for 24 h, and then treated with 10 μg/ml cycloheximide (CHX) for 2 h before the addition of 10 nM ouabain. Cells without any treatment or only treated with CHX were used as control. The cells were collected at different time points and lysed in RIPA buffer. Equal amount of protein was loaded to analyze the Na/K-ATPase by Western blot. The representative Western blot and the quantification data from 4 experiments were shown. The Na/K-ATPase α1 amount at each time point was normalized against the control at the same time point.

FIG. 6C: BT20 cells were treated as in panel B and assayed for Na/K-ATPase α1. *p<0.05.

FIGS. 7A-7D: Ouabain up-regulates Na/K-ATPase expression and cell growth through activation of PI3 kinase:

FIG. 7A: LLC-PK1 cells were pre-treated with 10 nM rapamycin or 50 μM LY294002 for 30 min. Ouabain 10 nM was then added into the medium for additional 24 h. The cell lysates were analyzed for Na/K-ATPase α1 using Western blot.

FIG. 7: LLC-PK1 and BT20 cells were incubated with ouabain for 15 min, and the cell lysates were analyzed for phosphorylated Akt (pAkt).

FIG. 7C: DU145 cells were serum-starved and treated with ouabain or 10 nM IGF for 15 min. The pAkt was probed by Western blot.

FIG. 7D: LLC-PK1 cells were pre-treated with 10 nM rapamycin or 50 μM LY294002 for 30 min and then incubated with ouabain for an additional 24 h. Cell growth was then measured using MTT assay.

FIGS. 8A-8D: Signaling function of Na/K-ATPase is required for its repletion after ouabain treatment:

FIG. 8A: Na/K-ATPase expression level in SYF cells and SYF-Src cells after 500 nM and 1 μM ouabain treatment for 72 h.

FIG. 8B: Measurement of Na/K-ATPase α1 expression level after 5 and 10 nM ouabain treatment for 72 h in control cells (P11) and caveolin-1 knock-down cells (C2-9).

FIG. 8C: C2-9 cells were treated with different concentrations of ouabain for 15 min. The cell lysates were analyzed for pAkt.

FIG. 8D: MTT assay was performed to compare the cell growth in response to ouabain treatment between P11 and C2-9 cells.

### DETAILED DESCRIPTION OF THE PREFERRED EMBODIMENT

The present invention is based, in part, on the discovery that ouabain-induced changes in the expression of Na/K-ATPase dictate its growth regulation.

The present invention is also based in part on the discovery that ouabain-induced activation of PI3-K/Akt/mTOR/mTOR pathway is a key to ouabain-induced up-regulation of the Na/K-ATPase, and that inhibition of PI3-K/Akt/mTOR/mTOR pathway by rapamycin can change the ouabain response from growth stimulation to growth inhibition.

The present invention is also based in part the discovery that ouabain and other cardiotonic steroids can be used together with rapamycin and rapamycin-like drugs for treatment of cancer.

The present invention is also based in part on the discovery that the expression of Na/K-ATPase may be useful to assess the effectiveness of digitalis therapy of certain cancers or individual patient and can be useful in the targeting the Na/K-ATPase for developing new anti-cancer therapeutics.

The present invention is also based in part on the discovery that inhibition of Na/K-ATPase by means other than rapamycin can also sensitize the cells to ouabain- and other cardiotonic steroids-induced cell growth inhibition.

The present invention is also based in part on the discovery that the stimulatory or inhibitory effect of ouabain on cell growth depends on whether cells can activate PI3-K/Akt/mTOR pathway and replete cellular Na/K-ATPase against ouabain-induced endocytosis and subsequent degradation of the enzyme.

The present invention is also based in part on the discovery that inhibition of the PI3-K/Akt/mTOR pathway by rapamycin sensitizes ouabain-induced depletion of Na/K-ATPase and cell growth inhibition.

The present invention is also based, in part, on the inventor's discovery that reduction of cellular Na/K-ATPase by means other than ouabain can also stimulate the expression of cell cycle inhibitor p21cip, resulting in cell growth inhibition.

The present invention is also based, in part, on the inventors' discovery that these findings indicate that ouabain regulates cell growth by changing the expression of Na/K-ATPase.

The following sections describe different aspects and embodiments of the invention in more detail.

Ouabain regulates the cell growth and Na/K-ATPase expression in a cell-specific manner.

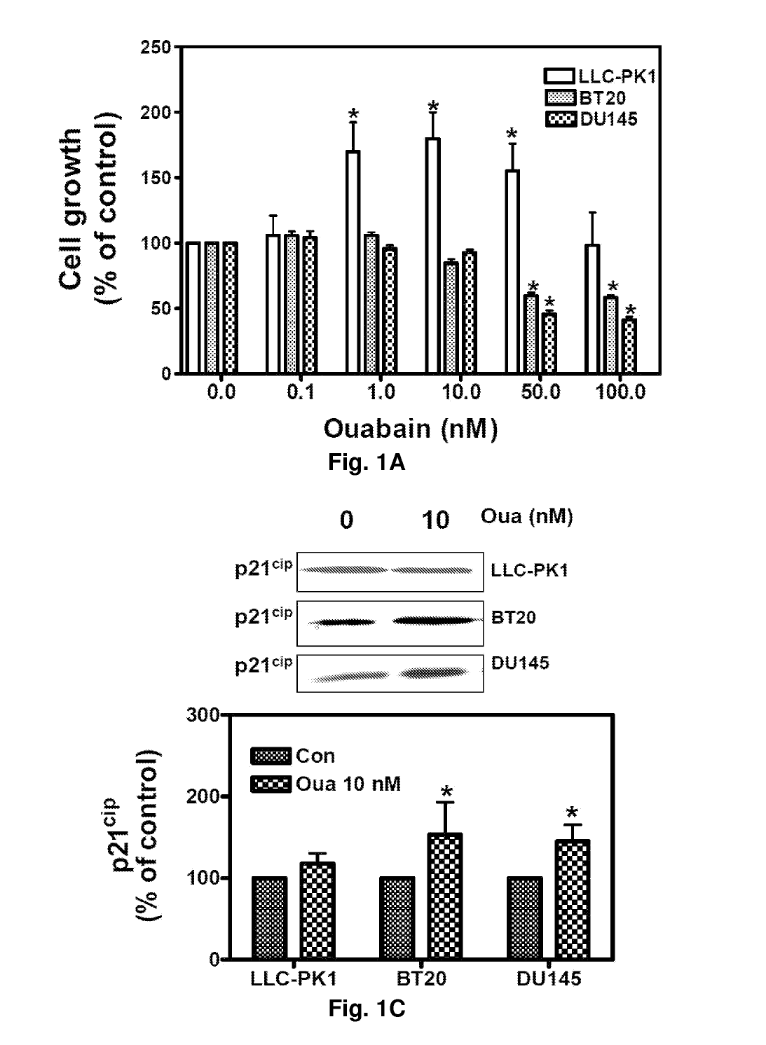

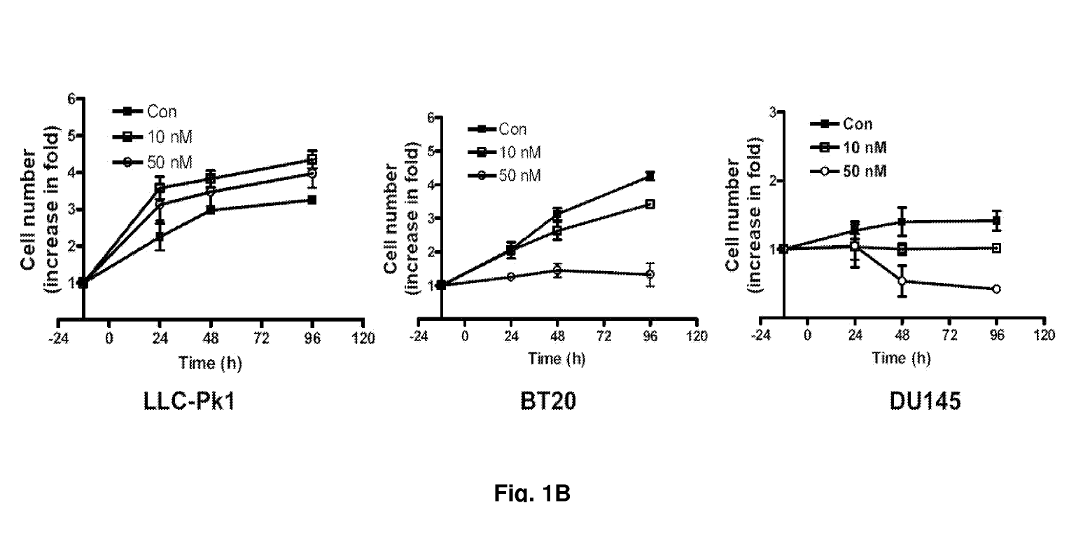

Ouabain can either stimulate or inhibit cell growth in a cell-specific manner. To understand the molecular mechanism of these opposite regulations, the inventors first compared the effects of ouabain on cell growth in two human epithelia-derived cancer cell lines (BT20 and DU145) and a pig kidney epithelial cell line (LLC-PK1). As shown in FIG. 1A, MTT assays indicated that ouabain at 1 to 50 nM stimulated cell growth in LLC-PK1 cells. These findings are consistent with the observed effects of ouabain on other renal epithelial cells. In contrast, the inventors failed to detect any stimulatory effect of ouabain on either breast cancer BT20 or prostate cancer DU145 cells. Moreover, 10-50 nM ouabain caused a significant inhibition of cell growth (FIG. 1A).

To further confirm these observations, the inventors determined the effects of 10 and 50 nM ouabain on cell growth as a function of time. As depicted in FIG. 1B, ouabain increased the number of LLC-PK1 cells in a time-dependent manner, confirming the growth stimulatory effects of ouabain on these cells.

On the contrary, the same treatment significantly reduced the number of BT20 cells. In fact, 10 and 50 nM ouabain appeared to be sufficient to completely block the growth of DU145 and BT20 cells, respectively. Because ouabain inhibited cell growth by stimulating the expression of a cell cycle inhibitor p21cip in human breast cancer MDA-MB-435s cells, the inventors measured the effects of ouabain on p21cip expression in BT20 and LLC-PK1 cells.

As depicted in FIG. 1C, while 10 nM ouabain stimulated the expression of cell cycle inhibitor p21cip in BT20 and DU145 cells, it failed to do so in LLC-PK1 cells.

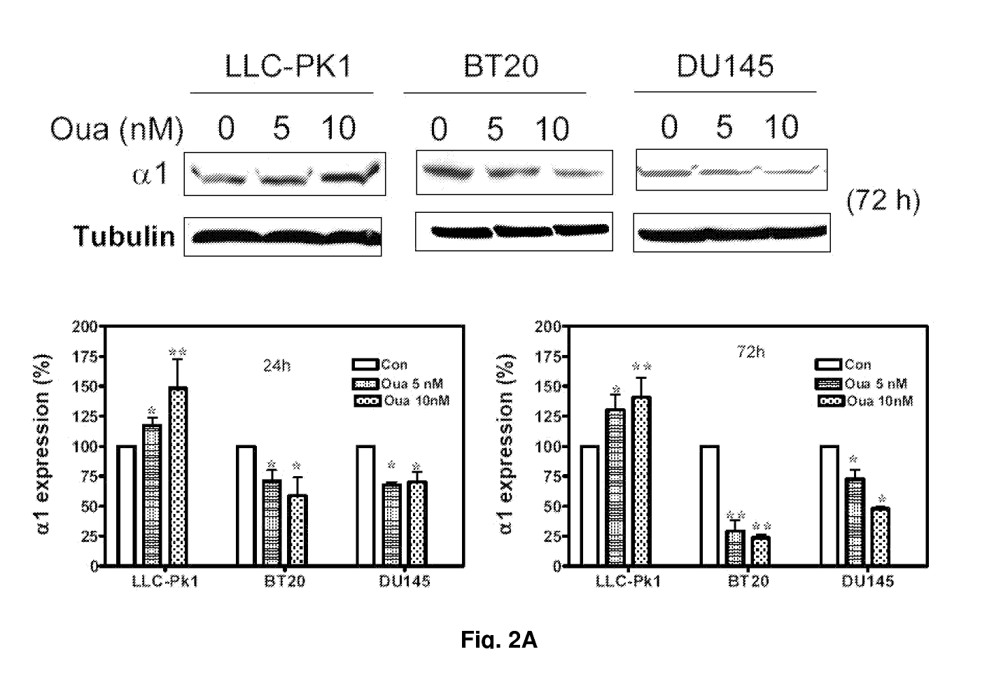

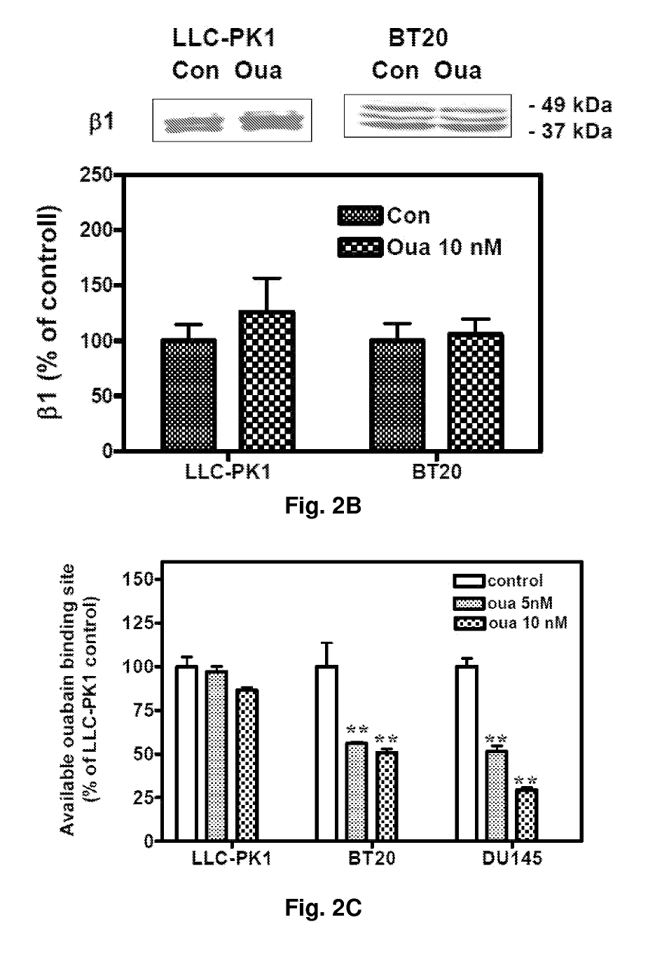

Since the Na/K-ATPase is the only known receptor for ouabain, the inventors next tested if ouabain affects the expression of Na/K-ATPase. Western blot analyses indicated that all three cell lines expressed a ouabain-sensitive α1 isoform of Na/K-ATPase (α1). Under the same experimental conditions, the expression of α2 and α3 isoforms was undetectable (data not shown). Interestingly, the effects of ouabain on α1 expression were also cell-specific. While ouabain caused an induction of α1 expression in LLC-PK1 cells after either 24 h or 72 h exposure, it reduced the cellular amount of α1 in both BT20 and DU145 cells (FIG. 2A).

While the effects of ouabain on α1 expression reached plateau after 24 h exposure in both LLC-PK1 and DU145 cells, further reduction in al expression was noted after 72 h of ouabain exposure in BT-20 cells. Because ouabain inhibited the growth of MDA-MB-435s cells, the inventors also measured the effects of ouabain on al expression after these cells were exposed to 5 nM ouabain for 24 h. A significant reduction in α1 expression was detected (82±2% of control, N=3, P<0.05). Thus, ouabain-induced changes in α1 expression are clearly correlated with its effects on cell growth.

The β1 subunit plays an important role not only in the formation of a functional

Na/K-ATPase, but also in the formation of tight junctions and in suppression of tumor cell growth. Thus, the inventors assessed whether ouabain affects β1 expression in LLC-PK1 and cancer cell lines. Cells were treated with 10 nM ouabain for 24 h and the amount of β1 was measured in the cell lysates by Western blot. As depicted in FIG. 2B, ouabain caused a modest increase in β1 expression in LLC-PK1, but not BT-20, cells.

To assess the functional consequence of these changes in α1 expression, the inventors measured the number of functional Na/K-ATPase in the plasma membrane. Cells were exposed to different concentrations of ouabain for 72h and then subjected to 3H-ouabain binding. It is important to note that this assay measured the number of Na/K-ATPase that was not occupied by the ouabain during 72 h treatment. As depicted in FIG. 2C, ouabain up to 10 nM barely changed the number of binding sites in LLC-PK1 cells. These data show that the ouabain-induced up-regulation of α1 is just sufficient to replete the plasma membrane pool of functional Na/K-ATPase. In contrast, ouabain caused a significant decrease in surface active Na/K-ATPase in both BT20 and DU145 cells (FIG. 2C). Taken together, the inventors herein now show that that the decrease in the plasma membrane Na/K-ATPase may be a key to the ouabain-induced growth inhibition in cancer cells.

Reduction of the Na/K-ATPase slows cell growth and sensitizes the cells to ouabain-induced cell growth inhibition.

Since LLC-PK1 cells and two cancer cell lines are derived from different species, in order to determine whether a decrease in cellular Na/K-ATPase is a key to ouabain-induced cell growth inhibition, the inventors herein measured the effects of graded knockdown of the Na/K-ATPase on cell growth in LLC-PK1 cells. The knockdown A4-11 and PY17 cells expressed about 40% and 10% of the Na/K-ATPase in comparison to the control vector-transfected LLC-PK1 (P-11) cells, respectively.

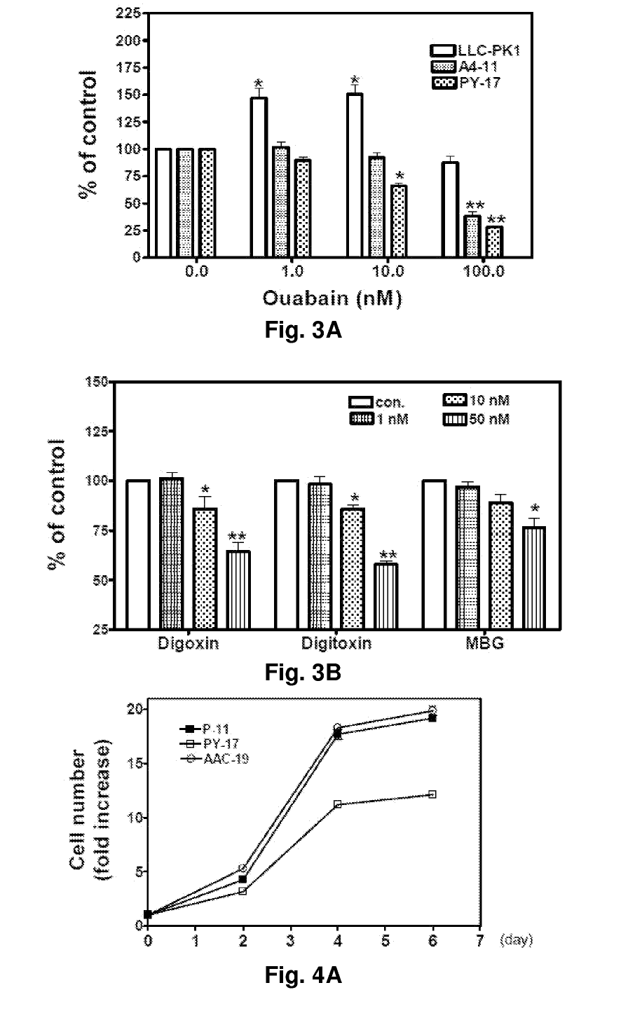

As shown in FIG. 3A, P-11 cells behaved like the parent LLC-PK1 cells in response to ouabain stimulation. Ouabain, at concentrations from 1 to 10 nM, stimulated the cell growth. In contrast, A4-11 and PY17 cells behaved like BT20 and DU145 cells. No growth stimulation was detected. Instead, reduction of Na/K-ATPase expression by siRNA-mediated mechanism was sufficient to sensitize these cells to ouabain-induced cell growth inhibition (FIG. 3A). For example, 10 nM ouabain caused more than 30% reduction in cell growth in PY-17 cells. Moreover, the growth inhibitory effects of ouabain were clearly correlated with the cellular amounts of Na/K-ATPase.

To test whether this growth-inhibitory effect of ouabain applies to other cardiotonic steroids, the inventors herein measured the effects of digoxin, digitoxin and marinobufagenin MGB on cell growth in PY-17 cells. As depicted in FIG. 3B, these compounds caused a similar growth inhibition as did ouabain.

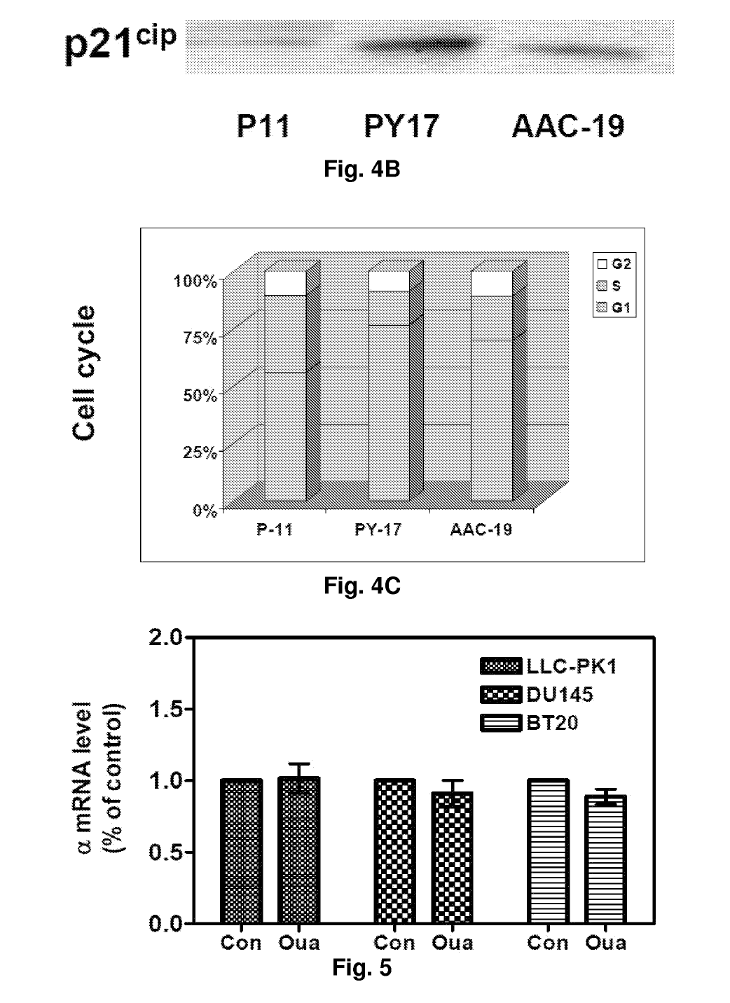

To further examine the role of Na/K-ATPase reduction in cell growth regulation, the inventors compared the cell growth curve of stable cell line P-11, PY-17 and AAC-19, a rat α1-rescued PY-17 cells. As shown in FIG. 4A, the Na/K-ATPase knock-down PY-17 cells grew much slower than the control P-11 cells, whereas the re-introduction of the rat Na/K-ATPase α1 restored the cell growth in AAC-19 cells.

Because ouabain stimulated the expression of p21cip and inhibited cell growth in human cancer cells (FIG. 1), the inventors herein determined whether knockdown of cellular Na/K-ATPase affects the expression of this cell cycle inhibitor. The expression of p21cip was quite low in the control P-11 cells (FIG. 4B). Knockdown of Na/K-ATPase caused a significant induction of this cell cycle inhibitor in PY-17 cells. Moreover, rescuing the PY-17 cells by knocking in rat α1 was sufficient to reduce the expression of p21cip in AAC-19 cells. Consistently, the inventors also detected more G0/G1 cells in PY-17 cells than that in both P-11 and AAC-19 cells (FIG. 4C).

Ouabain has no effect on al mRNA.

To probe the molecular mechanism of ouabain-induced cell-specific regulation of α1, the inventors first measured α1 mRNA using a semi-quantitative RT-PCR after the cells were treated with 10 nM ouabain. As depicted in FIG. 5, ouabain had no effect on levels of α1 mRNA in LLC-PK1, BT20 and DU145 cells. Thus, the ouabain-induced changes in α1 protein are likely via translational/post-translational mechanisms.

Ouabain stimulates endocytosis and degradation of Na/K-ATPase.

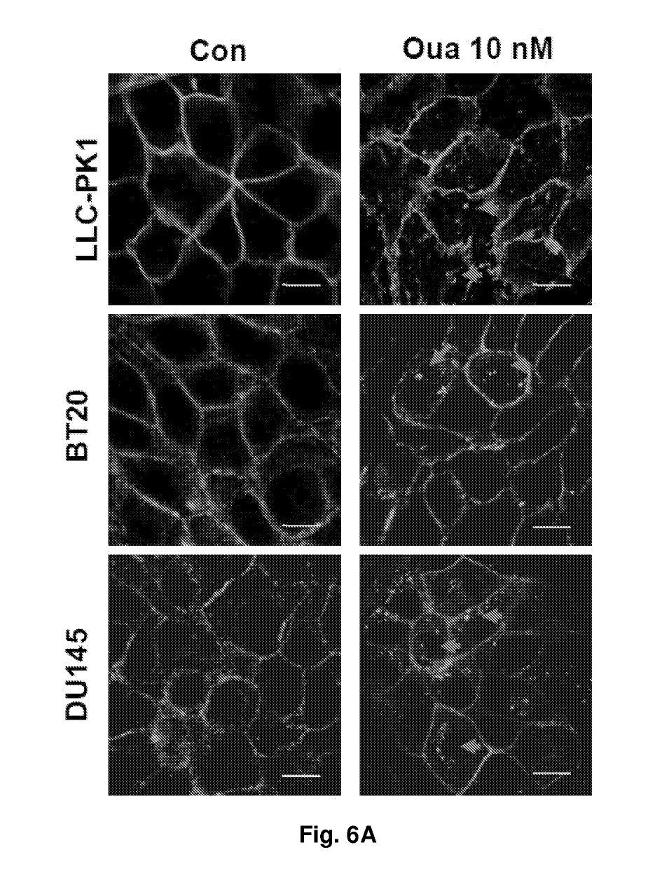

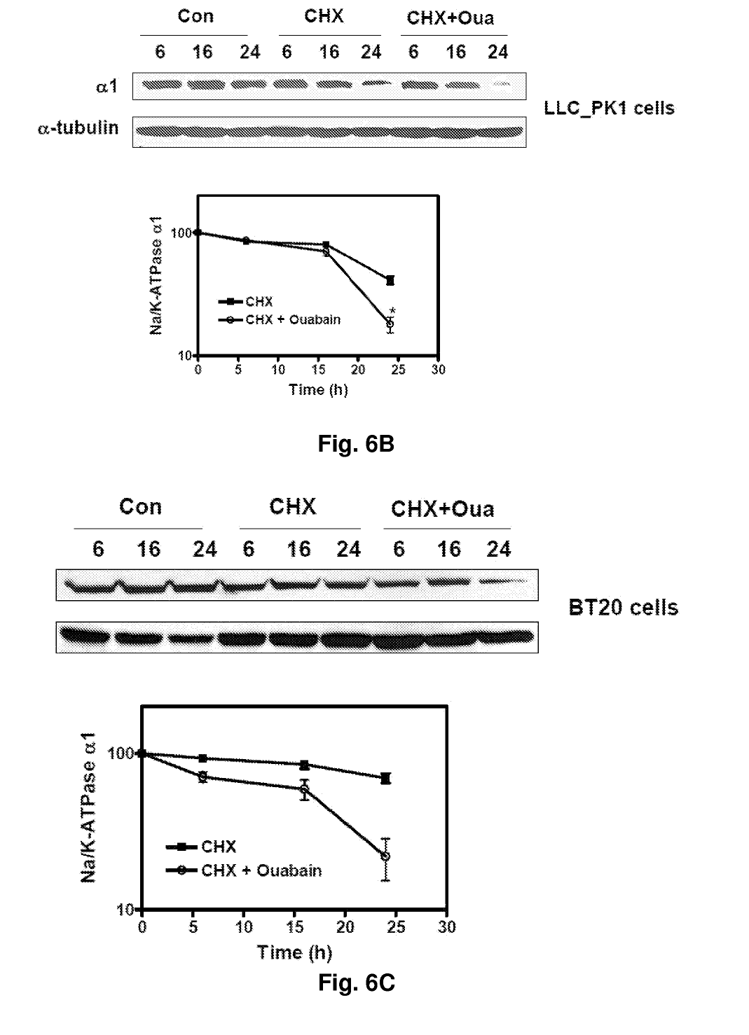

Ouabain stimulated Na/K-ATPase endocytosis in LLC-PK1 cells. If ouabain also stimulates the endocytosis in BT20 and DU145 cells, this may result in an increased degradation of Na/K-ATPase and a decrease in α1 protein in these cells. To test this, cells were exposed to 10 nM ouabain and then immunostained for α1. Most of α1 resided in the plasma membrane in BT20 and DU145 cells as in LLC-PK1 cells (FIG. 6A). After 24 h of ouabain exposure, many al-positive vesicles were accumulated in BT20 and DU145 cells.

To determine whether ouabain-induced endocytosis also leads to increased degradation of Na/K-ATPase, LLC-PK1 cells and BT-20 cells were pre-treated with cycloheximide, a protein synthesis inhibitor, for 1 h, and then exposed to 10 nM ouabain for 6, 16, and 24 h. As depicted in FIG. 6B and FIG. 6C, ouabain treatment produced more degradation of al subunit in both BT-20 and LLC-PK1 cells.

Ouabain activates Akt in LLC-PK1 cells, but not BT-20 and DU145 cells.

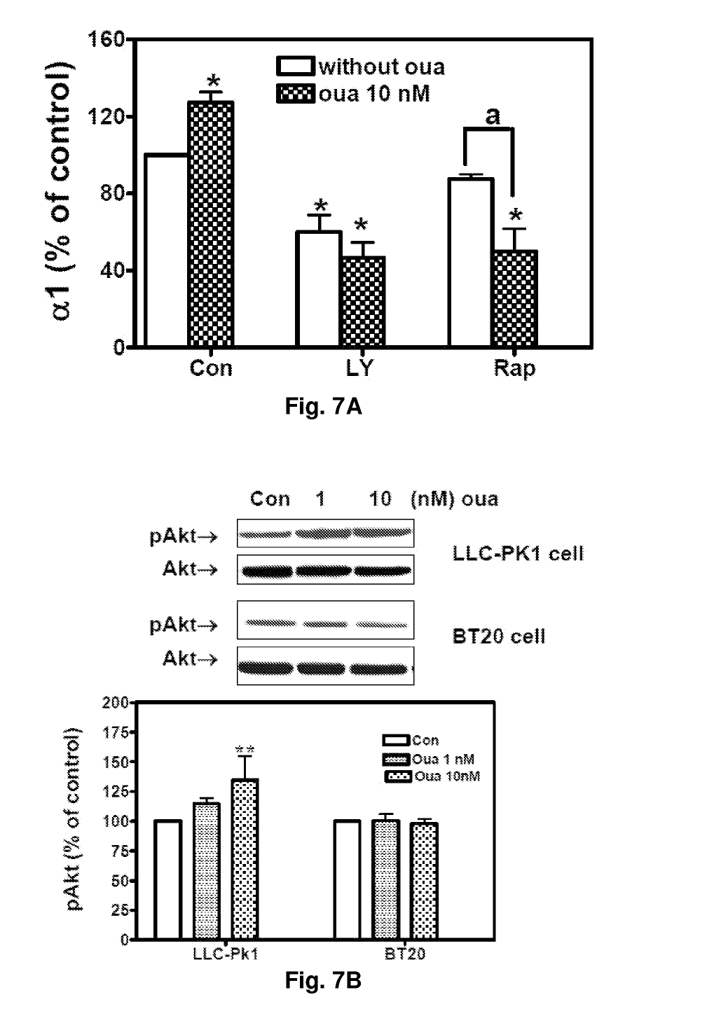

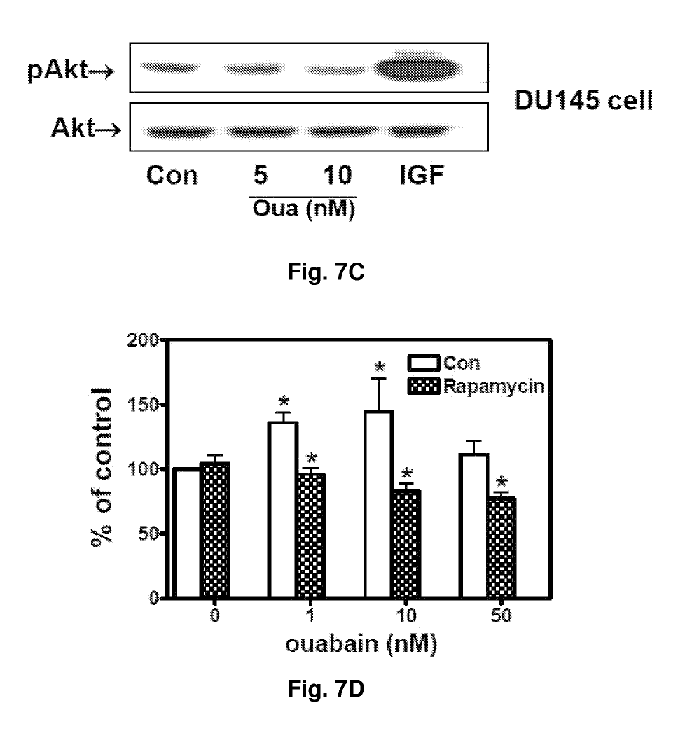

The inventors and others have demonstrated that ouabain stimulates PI3-K/Akt/mTOR pathway. The activation of mTOR may increase the translation of many mRNAs. Because ouabain did not increase al mRNA in LLC-PK1 cells, the inventors determined whether ouabain-induced up-regulation of al is sensitive to inhibitors of PI3-K (phosphoinositide 3-kinase) and mTOR. As depicted in FIG. 7A, pre-treatment of cells with either PI3-K inhibitor LY294002 or mTOR (mammalian target of rapamycin) inhibitor rapamycin completely abolished ouabain-induced up-regulation of al subunit. Both LY294002 and rapamycin reduced the basal expression of al. Moreover, in the presence of rapamycin, ouabain treatment actually resulted in a further decrease in the expression of al. While not wishing to be bound by theory, the inventors now believe that rapamycin may block ouabain-induced al expression but not ouabain-induced al endocytosis and degradation.

To test whether the activation of Akt/mTOR pathway is the key to ouabain-induced cell-specific regulation of al expression, the inventors next measured the effect of ouabain on Akt in three different cell lines. As shown in FIG. 7B and FIG. 7C, while ouabain stimulated Akt phosphorylation in LLC-PK1 cells, it failed to do so in both BT20 and DU145 cells. To be sure that the Akt pathway is intact in the cancer cells, the inventors exposed DU145 cells to IGF and measured for Akt activation. As shown in FIG. 7C, IGF caused a remarkable stimulation of Akt in DU145 cells. Consistently, when the same experiments were repeated in PY-17 cells, ouabain showed no effect on Akt phosphorylation (data not shown).

Rapamycin sensitizes LLC-PK1 cells to ouabain-induced cell growth inhibition.

The above data indicate that activation of PI3-K/Akt/mTOR is a key to ouabain-induced up-regulation of al. Because repletion of α1 is essential for ouabain-induced cell growth stimulation, the inventors then determined whether inhibition of mTOR by rapamycin would convert ouabain-induced cell growth into growth inhibition in LLC-PK1 cells. As shown in FIG. 7D, addition of rapamycin was sufficient to block ouabain-induced cell growth in LLC-PK1 cells. Moreover, it also sensitized the cell to ouabain-induced cell growth inhibition. For example, 10 nM ouabain was sufficient to cause a significant inhibition of cell growth in LLC-PK1 cells pre-treated with the inhibitor.

Involvement of Src and caveolin-1.

The Na/K-ATPase signals from caveolae by forming a receptor complex with Src.

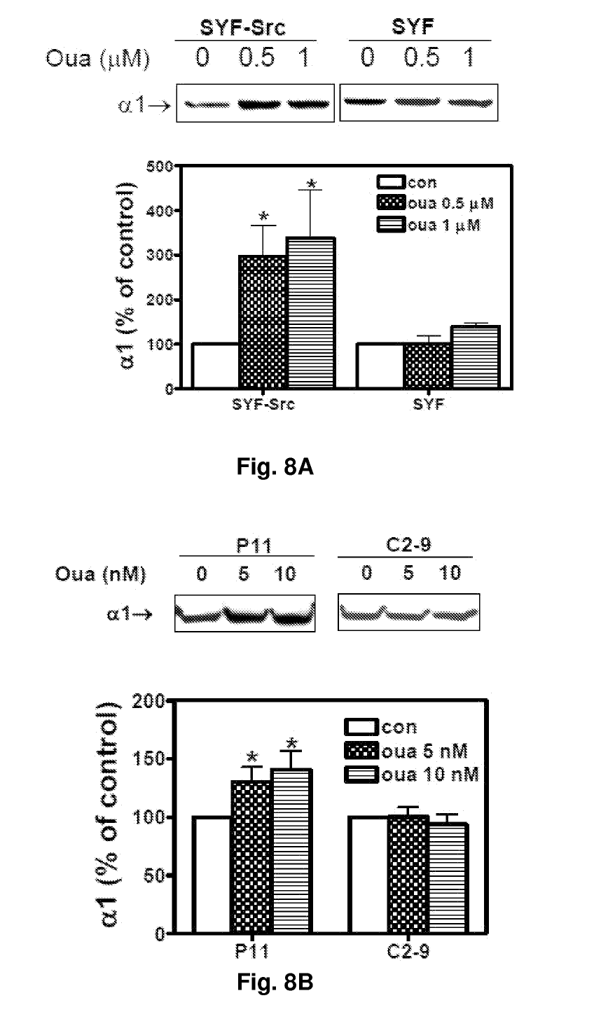

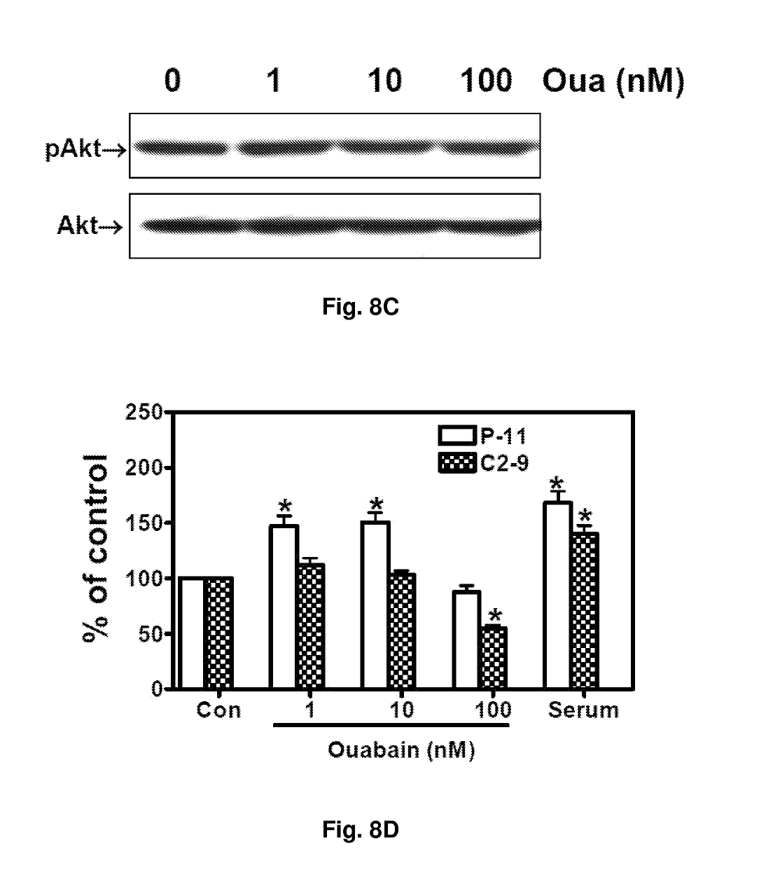

To further understand the molecular mechanism of ouabain-induced up-regulation of α1, the inventors first addressed the role of Src. SYF cells (in which the Src family kinase Src, Yes, and Fin were knocked out) were exposed to ouabain for 24 h. Because these cells were from mouse, 0.5 and 1.0 μM ouabain were used in the experiments. As depicted in FIG. 8A, knockout of Src completely blocked ouabain-induced α1 expression. Consistently, when SYF cells were rescued by c-Src, ouabain was able to cause a three-fold induction of α1 expression.

To test the involvement of caveolin-1 and caveolae, the inventors established a caveolin-1 knockdown LLC-PK1 cell line (C2-9 cells) that expressed about 20% of cavelin-1 in comparison to the parent LLC-PK1 cells because of the expression of caveolin-1-specific siRNA. As shown in FIG. 8B, knockdown of caveolin-1 completely abolished ouabain-induced increases in α1 expression. Consistently, ouabain failed to stimulate Akt in these cells (FIG. 8C). Moreover, knockdown of cavelon-1 not only abolished ouabain-induced cell growth stimulation, but also sensitized the cells to ouabain-induced cell growth inhibition (FIG. 8D).

Ouabain Stimulates Endocytosis and Degradation of Na/K-ATPase.

Ouabain stimulated Na/K-ATPase endocytosis in LLC-PK1 cells. If ouabain also stimulates the endocytosis in BT20 and DU145 cells, this could result in an increased degradation of Na/K-ATPase and a decrease in the a1 protein in these cells. The cells were exposed to 10 nM ouabain and then immunostained for a1. Most of al resided in the plasma membrane in BT20 and DU145 cells as in LLC-PK1 cells (FIG. 9A). After 6 h of ouabain exposure, many al-positive vesicles were accumulated in BT20 and DU145 cells. To determine whether ouabain-induced endocytosis also leads to increased degradation of Na/K-ATPase, LLC-PK1 cells and BT-20 cells were pretreated with cycloheximide, a protein synthesis inhibitor, for 1 h, and then exposed to 10 nM ouabain for 6, 16, and 24 h. As shown in FIG. 9B and FIG. 9C, ouabain treatment produced more degradation of the a1 subunit in both BT-20 and LLC-PK1 cells.

Ouabain Activates Akt in LLC-PK1 but not BT-20 and DU145 Cells.

Ouabain stimulates the PI3-K/Akt/mTOR pathway. Activation of mTOR could increase the translation of many mRNAs. Because ouabain did not increase al mRNA in LLC- PK1 cells, the inventors herein tested whether ouabain-induced up-regulation of a1 is sensitive to inhibitors of PI3-K and mTOR. As shown in FIG. 10A, pretreatment of cells with either PI3-K inhibitor LY294002 or mTOR inhibitor rapamycin completely abolished ouabain-induced up-regulation of a1 subunit. Interestingly, both LY294002 and rapamycin reduced the basal expression of a1. Moreover, in the presence of rapamycin, ouabain treatment actually resulted in a further decrease in the expression of a1. While not wishing to be bound by theory, the inventors herein now believe that rapamycin may block ouabain-induced a1 expression but not ouabain-induced a1 endocytosis and degradation. To test whether activation of the Akt/mTOR pathway is involved in ouabain-induced cell-specific regulation of a1 expression, the inventors herein measured the effect of ouabain on Akt in three different cell lines. Whereas ouabain stimulated Akt phosphorylation in LLC-PK1 cells, it failed to do so in both BT20 and DU145 cells. To be sure that the Akt pathway is intact in cancer cells, the inventors herein we exposed DU145 cells to IGF and measured for Akt activation. IGF caused a remarkable stimulation of Akt in DU145 cells. Consistently, when the same experiments were repeated in PY-17 cells, ouabain showed no effect on Akt phosphorylation (data not shown).

Rapamycin Sensitizes LLC-PK1 Cells to Ouabain-induced Cell Growth Inhibition.

The above data indicate that activation of PI3-K/Akt/mTOR is required for ouabain to increase the expression of a1. Because repletion of a1 is essential for ouabain-induced cell growth stimulation, the inventors herein determined that inhibition of mTOR by rapamycin would convert ouabain-induced cell growth into growth inhibition in LLC-PK1 cells. As shown in FIG. 10D, the addition of rapamycin was sufficient to block ouabain-induced cell proliferation in LLC-PK1 cells. Moreover, it also sensitized the cell to ouabain-induced cell growth inhibition. For example, 10 nM ouabain was sufficient to cause significant inhibition of cell proliferation in LLCPK1 cells pretreated with the inhibitor.

Discussion

The stimulatory or inhibitory effect of ouabain on cell growth depends on whether cells can activate Akt/mTOR pathway and replete cellular Na/K-ATPase against ouabain-induced endocytosis and subsequent degradation of the enzyme.

The inhibition of the Akt/mTOR pathway by rapamycin sensitizes ouabain-induced depletion of Na/K-ATPase and cell growth inhibition.

The reduction of cellular Na/K-ATPase by means other than ouabain can also stimulate the expression of cell cycle inhibitor p21cip, resulting in cell growth inhibition. Thus, these findings indicate that ouabain regulates cell growth by changing the expression of Na/K-ATPase.

Translational up-regulation of Na/K-ATPase by ouabain depends on the PI3-K/Akt/mTOR pathway: The Na/K-ATPase is an important ion pump that converts ATP into transmembrane ion gradients. It also interacts with Src to form a receptor complex. While the Na/K-ATPase provides the binding site for cardiotonic steroids, the Na/K-ATPase-associated Src acts as a signal transducer, converting the ligand binding to stimulation of protein kinase cascades and the generation of second messengers. Although ouabain exhibited both stimulatory and inhibitory effects on cell growth in a cell-specific manner, the activation of proximal signaling events (e.g., Src and ERK) have been recorded in both types of cells. Moreover, ouabain induced the endocytosis and subsequent degradation of Na/K-ATPase in cells that could either be stimulated or inhibited by ouabain.

The inventors herein now believe that the ouabain-activated downstream events must diverge significantly in cells whose growth response to ouabain went to opposite directions. In accordance therewith, the inventors have now observed that ouabain stimulated the PI3-K/Akt/mTOR pathway, thus resulting in translational up-regulation of Na/K-ATPase and cell growth stimulation in LLC-PK1 cells. In contrast, ouabain failed to activate the PI3-K/Akt/mTOR pathway and replete the Na/K-ATPase in human cancer cells such as BT20, MDA-MB-435s and DU145 cells as well as in the Na/K-ATPase-knockdown cells (FIG. 7). Thus, activation of the PI3-K/Akt/mTOR pathway may be a key to ouabain-induced cell growth stimulation. This is in accordance with the note that ouabain-induced activation of PI3-K/Akt/mTOR pathway has been reported by others in several cell lines and primary cultures. Also, this activation appears to be required for ouabain to stimulate growth in these cells.

Caveolae compartmentalize signaling events. Specifically, disruption of caveolae by depletion of either caveolin-1 or cholesterol can significantly alter insulin/IGF-induced activation of PI3-K/Akt/mTOR pathways. Consistently, Cavi knockout causes insulin resistance in vivo. Also, the expression of caveolin-1 and/or Na/K-ATPase is significantly reduced in many cancer cell lines. Specifically, the expression of caveolin-1 is almost absent in BT20 cells, while the expression of Na/K-ATPase is significantly reduced in DU145 cells. Because both caveolin-1 and Na/K-ATPase are important for the formation of caveolae, the inventors herein now believe that reduction of either caveolin-1 or the Na/K-ATPase may contribute to the uncoupling of ouabain binding to the activation of PI3-K/Akt/mTOR pathway in these cells. Consistently, as shown in FIG. 8, the inventors herein found that knockdown of caveolin-1 or the Na/K-ATPase indeed abolished ouabain-induced cell growth response in LLC-PK1 cells. Moreover, it also blocked ouabain-induced activation of Akt.

Large increases in intracellular Na+ are sufficient to stimulate a transcriptional mechanism and up-regulate the expression of Na/K-ATPase. This occurs when cells are exposed to low extracellular K+ or ouabain concentrations that produce more than 50% inhibition of Na/K-ATPase. Apparently, this is unlikely the molecular mechanism by which 1-10 nM ouabain increased the expression of Na/K-ATPase in LLC-PK1 cells. First, ouabain at 1-10 nM does not affect cellular Na+ concentration in LLC-PK1 cells. Second, the ouabain-induced up-regulation is different from that of low K+ because no change in α1 mRNA was detected. On the other hand, activation of PI3-K/Akt/mTOR pathway can stimulate translational regulation of many mRNAs.

While not wishing to be bound by theory, the inventors herein now believe that ouabain at low concentrations increases the expression of the Na/K-ATPase via a PI3-K/Akt/mTOR-dependent translational mechanism. The inventors show herein that inhibition of the PI3-K/Akt/mTOR pathway by either LY294002 or rapamycin was sufficient to block ouabain-induced up-regulation of the Na/K-ATPase.

As an ion pump, the Na/K-ATPase maintains normal Na+ and K+ gradients across cell membrane. Significant alteration of these gradients by either knock out of Na/K-ATPase or by completely inhibiting the enzyme is lethal to mammalian cells. Hence, it may be that a modest decrease in cellular Na/K-ATPase could cause cell growth inhibition.

The inventors have now found that knockdown of Na/K-ATPase by siRNA was sufficient to stimulate the expression of p21^{cip}, resulting in growth inhibition in LLC-PK1 cells. Consistently, rescuing these cells by expression of a rat α1 was sufficient to inhibit the expression of p21cip and restore the cell growth. Interestingly, in many types of cells binding of ouabain and other cardiotonic steroids to the Na/K-ATPase is known to induce the endocytosis and degradation of the enzyme. Functionally, the ouabain-induced endocytosis, even by low concentrations of ouabain, could lead to a significant reduction of Na/K-ATPase over the time if it is not compensated by up-regulation of al expression. In principle, this down regulation may be sufficient to account for ouabain-induced cell growth arrest. This notion is supported by the fact that low concentrations of ouabain, over the time, were capable of inducing the expression of p21cip and inhibiting the growth of breast cancer cells. Significantly, blocking ouabain-induced repletion of Na/K-ATPase by rapamycin abolished ouabain-induced cell growth stimulation in LLC-PK1 cells. It also sensitized these cells to ouabain-induced cell growth inhibition. Taken together, it is clear that the amount of cellular Na/K-ATPase is important for cell growth control.

The epidemiological studies in late 70s and early 80s indicated the potential benefits of using digitalis drugs in patients with breast cancer. Over the years, the effectiveness of digitalis drugs such as digoxin and digitoxin as well as new derivatives of this class of compounds have been demonstrated in cell cultures and in animal models. Significantly, phase I and II clinic trials are underway in the US and in Europe to test these new compounds as anti-cancer drugs. Because these compounds also possess well-documented growth-stimulatory effects on various cells both in vitro and in vivo, it is important to identify the divergent pathways that are responsible for this opposite growth regulation.

To this end, the inventors herein now show that ouabain-induced changes in the expression of Na/K-ATPase dictate its growth regulation. Moreover, the inventors identify the ouabain-induced activation of PI3-K/Akt/mTOR pathway as a key to ouabain-induced up-regulation of the Na/K-ATPase. In addition, the inventors show that inhibition of PI3-K/Akt/mTOR pathway by rapamycin, a widely used drug, can change the ouabain response from growth stimulation to growth inhibition.

The expression of Na/K-ATPase may be useful to assess the effectiveness of digitalis therapy of certain cancers or individual patient. Moreover, these results indicate the usefulness of targeting the Na/k-ATPase for developing new anti-cancer therapeutics. Also, these results indicate a therapeutic utility of rapamycin in digitalis therapy of cancer.

Exemplary Embodiments

The following examples are for illustrative purpose only, and should in no way be construed to be limiting in any respect of the claimed invention.

Materials: Cell culture media, fetal bovine serum, and trypsin were purchased from

Invitrogen (Burlington, ON); Optitran nitrocellulose membrane was from Schleicher & Schuell (Keene, NH); Enhanced chemiluminescence (ECL) super signal kit was purchased from Pierce (Rockford, Ill.). The monoclonal anti-al antibody (α6F) was obtained from the Developmental Studies Hybridoma Bank at the University of Iowa. The anti-p21cip1 antibody, anti-Src monoclonal antibody and all the secondary horseradish peroxidase conjugated antibodies were from Santa Cruz (Santa Cruz, Calif.). The polyclonal anti-Akt and anti-pAkt at Ser473 antibodies were from Cell Signaling (Danvers, Mass.). The MTT assay kit was purchased from American Type Culture Collection (ATCC) (Manassas, Va.). 3H-ouabain was from NEN Life Science Products (Boston, Mass.).

Cell culture: The pig kidney epithelia cells (LLC-PK1 cells), human breast cancer cells (BT-20 cells), and human prostate cancer cells (DU145 cells) were obtained from ATCC and maintained in Dulbecco's modified Eagle's medium (DMEM) or RPMI 1640 medium (for DU145 cells) in the presence of 10% fetal bovine serum (FBS), 100-units/ml penicillin, and 100 μg/ml streptomycin in 5% CO2 humidified incubator. The Na/K-ATPase knock-down (A4-11 and PY17) and caveolin-1 knock-down (C2-9) cells were derived from LLC-PK1 cells and cultured in DMEM. Cells were serum-starved for 24 h before experiments unless otherwise indicated.

Western blot analysis: Cells were washed with PBS and solubilized in modified and ice-cold RIPA buffer. The cell lysates were then centrifuged at 14,000 rpm and the supernatant was used for protein assay and subjected to Western blot analysis. Membranes were blocked with 4% milk in TBS-T (Tris-HCl 10 mM, NaCl 150 mM, Tween 20, 0.05%; pH 8.0) for 1 h at room temperature and probed with specific antibodies. Protein signals were detected using ECL kit and quantified using Bio-Rad GS-670 imaging densitometer.

MTT assay for measuring cell proliferation: Cells were sub-cultured in 96 well plates at 10,000 cells/well in 100 μl culture medium. After 12 h serum starvation, cells were exposed to different concentrations of ouabain for 24 h. Ten μl MTT reagent (3-4,5-dimethylthiazol-2-yl)-2,5-diphenyltetrazolium bromide) was added to each well for 90 min. The formazan crystal was dissolved with detergent and the OD value was measured at 570 nm.

Cell growth curve and cell cycle measurement: Cells were sub-cultured in 12 well plates at 50,000/well in 1 ml culture medium for 24 h. The cells were then serum-starved for 24 h and treated with different concentration of ouabain for indicated time periods. At each indicated time point, three wells of control or treated cells were trypsinized and counted. For cell cycle measurement, cells were trypsinized and re-suspended in Citrate buffer (8.55% sucrose, 1.18% Na citrated, 5% DMSO, pH 7.6), and stained with propidium iodide (PI). The stained cells were then analyzed by Flow Cytometry.

3H-ouabain binding: To measure effects of ouabain on cell surface expression of Na/K-ATPase, cells were cultured in 12-well plates and treated with ouabain for 72 h. Afterwards, cells were washed with serum-free medium and subjected to 3H-ouabain binding assay. Briefly, cells were incubated in K+-free Kreb's solution (NaCl 137 mM; KCl 0 mM; CaCl2 2.8 mM; NaH2PO4 0.6 mM; MgSO4 1.2 mM; dextrose; 10 mM; Tris 15 mM; pH7.4) for 15 min, and then exposed to 100 nM 3H-ouabain for 30 min at 37° C. At the end of incubation, the cells were washed 3 times with ice-cold K+-free Kreb's solution, solubilized in 0.1M NaOH-0.2% SDS, and counted in a scintillation counter for 3H-ouabain. Non-specific binding was measured in the presence of 5 mM unlabeled ouabain and substracted from total binding. Paralleled plates were used for counting cell numbers. 3H-ouabain binding data were calculated as binding sites per cell.

Confocal imaging and immunocytochemistry: LLC-PK1, BT20, and DU145 cells were serum-starved for 24 h and treated with ouabain (10nM) for 6 h on coverslips. The cells were fixed with ice-cold methanol for 15 min and blocked with Signal Enhancer from Invitrogen. Monoclonal anti-Na/K-ATPase α1 antibody was then incubated with the cells overnight at 4° C. After 3 washes, a secondary Alexa 488-conjugated anti-mouse antibody was added and incubated for 2 h at room temperature. The coverslip was washed, mounted and imaged using a Leica confocal microscope.

Quantitative RT-PCR: The cells were seeded in 6-cm dishes and serum-starved for 24 h after the confluence reached 90%. The cells were then treated with 10 nM ouabain for 24 h. Total RNA was extracted using TRIzol and subjected to quantitative RT-PCR analysis. The assays were performed on the AB 7500 real time PCR system with the following protocol: 1 cycle (6 min at 95° C.), 40 cycles (30 s at 95° C., 30 s at 52° C., 30 s at 72° C., and 1 min at 85° C.), followed by a dissociation stage to ensure a single product. The reactions are in a total volume of 25 μl, including 1.25 pl of Sybr Green (Invitrogen), 0.125 pl of DNA polymerase (Denville), 2.5 pl of dNTP (Invitrogen), 10 ng of cDNA. Primer pairs were used at 5 μM concentration, and the sequences are:

human GAPDH,
[SEQ ID NO: 1]
5′-GGGAAGGTGAAGGTCGGAGT
and
[SEQ ID NO: 2]
3′-TCCACTTTACCAGAGTTAAAAGCAG;
human Na/K-ATPase a1,
[SEQ ID NO: 3]
5′-TGTCCAGAATTGCAG
and
[SEQ ID NO: 4]
3′-TGCCCGCTTAAGAATAGGTAGGT;
pig GAPDH,
[SEQ ID NO: 5]
5′-ATGCTGGTGCTGAGTTCGTGG
and
[SEQ ID NO: 6]
3′-AGATGATGACCCTTTTGGCTCC;
and
pig Na/K-ATPase a1,
[SEQ ID NO: 7]
5′-ATCTCTGCTTCGTTGGGCTCATCT
and
[SEQ ID NO: 8]
3′-AATGGCTTTGGCTGTGATGGGATG.

The relative Na/K-ATPase al gene expression was calculated by comparative threshold (C_{T}) method (ΔΔ_{CT}). To normalize the difference amounts of cDNA, GAPDH was used as an internal control.

Statistics: Data are presented as Mean±SEM and compared using Student's t-test.

Significance was accepted at p<0.05.

Exemplary Treatment Methods: The method of present invention is advantageous over combination therapies known in the art because it allows conventional anti-cancer agent to exert greater effect at lower dosage. In certain non-limiting examples, an effective dose (ED50) for an anti-cancer agent or combination of conventional anti-cancer agents when used in combination with ouabain can be at least 2-fold less than the ED50 for the anti-cancer agent alone. Also, in certain non-limiting embodiments, the therapeutic index (TI) for such anti-cancer agent or combination of such anti-cancer agent when used in combination with CTS, such as ouabain, digoxin or digitoxin, can be at least 2-fold greater than the TI for conventional anti-cancer agent regimen alone.

Treatment Methods: In yet other embodiments, the method combines ouabain with other therapies such as chemotherapies and/or radiation therapies, including ionizing radiation, gamma radiation, or particle beams.

Administration and Therapy: The methods of the present invention may also comprise initially administering to the subject an antitumor agent so as to render the neoplastic cells in the subject resistant to an antitumor agent and subsequently administering an effective amount of any of the compositions of the present invention, effective to selectively induce terminal differentiation, cell growth arrest and/or apoptosis of such cells, or to treat cancer or provide chemoprevention.

Dosages and Dosage Schedules: The dosage regimen can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Non-limiting examples of suitable dosages can include total daily dosage of between about 25-4000 mg/m2 administered orally once-daily, twice-daily or three times-daily, continuous (every day) or intermittently (e.g., 3-5 days a week). For example, the compositions can be administered in a total daily dose, or divided into multiple daily doses such as twice daily, and three times daily.

In addition, the administration can be continuous, i.e., every day, or intermittently. The terms “intermittent” or “intermittently” as used herein means stopping and starting at either regular or irregular intervals. For example, intermittent administration may be administration one to six days per week or it may mean administration in cycles (e.g., daily administration for two to eight consecutive weeks, then a rest period with no administration for up to one week) or it may mean administration on alternate days.

In addition, the compositions may be administered according to any of prescribed schedules, consecutively for a few weeks, followed by a rest period. For example, the composition may be administered according to any one of the prescribed schedules from two to eight weeks, followed by a rest period of one week, or twice daily at a dose for three to five days a week.

It should be apparent to a person skilled in the art that the various dosages and dosing schedules described herein merely set forth specific embodiments and should not be construed as limiting the broad scope of the invention. Any permutations, variations and combinations of the dosages and dosing schedules are included within the scope of the present invention.

Pharmaceutical Compositions: The compounds of the invention, and derivatives, fragments, analogs, homologs pharmaceutically acceptable salts or hydrate thereof, can be incorporated into pharmaceutical compositions suitable for oral administration, together with a pharmaceutically acceptable carrier or excipient. Such compositions typically comprise a therapeutically effective amount of any of the compounds described herein, and a pharmaceutically acceptable carrier. Preferably, the effective amount is an amount effective to selectively induce terminal differentiation of suitable neoplastic cells and less than an amount which causes toxicity in a patient.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent (e.g., croscarmellose sodium) and a lubricant (e.g., magnesium stearate), and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

The pharmaceutical compositions can be administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like.

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Non-limiting examples of solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Non-limiting examples of liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In certain embodiments, the active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. For example, the compounds may be administered intravenously on the first day of treatment, with oral administration on the second day and all consecutive days thereafter. The compounds of the present invention may be administered for the purpose of preventing disease progression or stabilizing tumor growth.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the ouabain administered to the patient is less than an amount that would cause toxicity in the patient. In the certain embodiments, the amount of the compound that is administered to the patient is less than the amount that causes a concentration of the compound in the patient's plasma to equal or exceed the toxic level of the compound. Preferably, the concentration of the compound in the patient's plasma is maintained at about 10 nM. In another embodiment, the concentration of the compound in the patient's plasma is maintained at about 25 nM. In another embodiment, the concentration of the compound in the patient's plasma is maintained at about 50 nM.

In another embodiment, the concentration of the compound in the patient's plasma is maintained at ranges between about 10 to about 50 nM. The optimal amount of the compound that should be administered to the patient in the practice of the present invention will depend on the particular compound used and the type of cancer being treated.

In Vitro Methods: The present invention also provides in-vitro methods for selectively inducing terminal differentiation, cell growth arrest and/or apoptosis of neoplastic cells thereby inhibiting proliferation of such cells, by contacting the cells with an effective amount of a composition containing ouabain, or a pharmaceutically acceptable salt or hydrate thereof.

Although the methods of the present invention can be practiced in vitro, it is contemplated that the preferred embodiment for the methods of selectively inducing terminal differentiation, cell growth arrest and/or apoptosis of neoplastic cells, and the like will comprise contacting the cells in vivo, i.e., by administering the compounds to a subject harboring neoplastic cells or tumor cells in need of treatment.

Thus, the present invention also provides methods for selectively inducing terminal differentiation, cell growth arrest and/or apoptosis of neoplastic cells in a subject, thereby inhibiting proliferation of such cells in the subject, by administering to the subject a pharmaceutical composition comprising an effective amount of ouabain, or a pharmaceutically acceptable salt or hydrate thereof, and a pharmaceutically acceptable carrier or diluent.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1-41. (canceled)

42. A composition for modulating cellular synthesis of Na/K-ATPase, comprising: i) a cardiotonic steroid (CTS), and ii) a mTOR inhibitor and/or phosphoinisitide 3-kinase (PI3-K) inhibitor, in amounts effective to decrease cellular Na/K-ATPase in the patient in need thereof.

43. The composition of claim 42, wherein the cardiotonic steroid (CTS) is selected from one or more of: cardenolides and bufadienolides.

44. The composition of claim 42, wherein the cardiotonic steroid (CTS) comprises a cardenolides selected from one or more of: ouabain, digoxin and digitoxin.

45. The composition of claim 42, wherein the cardiotonic steroid (CTS) comprises a bufadienolide such as marinobufagenin.

46. The composition of claim 42, wherein the mTOR inhibitor comprises: rapamycin or an analog thereof.

47. The composition of claim 42, formulated with a pharmaceutically acceptable excipient and suitable for use in human patients.

48. The composition of claim 42, comprising an effective amount of a Na/K-ATPase inhibitor so as to modulate the biological activity of Na/K-ATPase.

49. The composition of claim 42, comprising: about 1 to about 50 nM, cardiotonic steroid (CTS) or derivative thereof.

50. The composition of claim 42, comprising: about 1 to about 50 nM ouabain as the cardiotonic steroid (CTS).

51. The composition of claim 42, comprising: about 10 to about 50 nM ouabain as the cardiotonic steroid (CTS).

52. The composition of claim 42, comprising: about 10 nM ouabain as the cardiotonic steroid (CTS).

53. The composition of claim 42, comprising: about 5 nM ouabain as the cardiotonic steroid (CTS).

54. The composition of claim 42, comprising: about 0.5 to about 1.0 μM ouabain as the cardiotonic steroid (CTS).

55. A method for modulating cellular synthesis of Na/K-ATPase in a cancer cell, comprising: administering a composition comprised of: i) a cardiotonic steroid (CTS), and ii) a mTOR inhibitor and/or phosphoinisitide 3-kinase (PI3-K) inhibitor, in amounts effective to decrease cellular Na/K-ATPase in the patient in need thereof.

56. The method of claim 55, wherein the cardiotonic steroid (CTS) is selected from one or more of: cardenolides and bufadienolides.

57. The method of claim 55, wherein the cardiotonic steroid (CTS) comprises a cardenolides selected from one or more of: ouabain, digoxin and digitoxin.

58. The method of claim 55, wherein the cardiotonic steroid (CTS) comprises a bufadienolide such as marinobufagenin.

59. The method of claim 55, wherein the mTOR inhibitor comprises:
rapamycin or an analog thereof.

60. The method of claim 55, formulated with a pharmaceutically acceptable excipient and suitable for use in human patients.

61. The method of claim 55, comprising an effective amount of a Na/K-ATPase inhibitor so as to modulate the biological activity of Na/K-ATPase.

62. The method of claim 55, comprising: about 1 to about 50 nM, cardiotonic steroid (CTS) or derivative thereof.

63. The method of claim 55, comprising: about 1 to about 50 nM ouabain as the cardiotonic steroid (CTS).

64. The method of claim 55, comprising: about 10 to about 50 nM ouabain as the cardiotonic steroid (CTS).

65. The method of claim 55, comprising: about 10 nM ouabain as the cardiotonic steroid (CTS).

66. The method of claim 55, comprising: about 5 nM ouabain as the cardiotonic steroid (CTS).

67. The method of claim 55, comprising: about 0.5 to about 1.0 μM ouabain as the cardiotonic steroid (CTS).

68. The method of claim 55, wherein the cancer cell is a breast cancer cell.

69. The method of claim 55, wherein the cancer cell is a prostate cancer cell.

70. The method of claim 55, wherein the cancer cell is ex vivo or in vivo.

71. A method for determining whether a cancer cell is susceptible to a chemotherapeutic treatment, comprising:
treating the cancer cell with a cardiotonic steroid (CST), and
determining whether the cancer cell loses the ability to increase cellular synthesis of Na/K-ATPase,
wherein a loss of Na/K-ATPase synthesis indicates that the cancer cell may benefit from the CTS treatment, and,
wherein if there is no loss of Na/K-ATPase synthesis, then
determining whether the cancer cell lacks a mechanism to compensate for the CST-induced Na/K ATPase loss.

72. The method of claim 72, includes treating the cancer cell with a CTS and a mTOR inhibitor or phosphoinisitide 3-kinase (PI3-K) inhibitor.

73. The method of claim 72, including detecting PI3-Kinase activation in the cancer cell which affects CST-induced Na/K-ATPase up-regulation in the cancer cell.

74. The method of claim 72, wherein the cancer cell is a breast cancer cell.

75. The method of claim 72, wherein the cancer cell is a prostate cancer cell.

76. The method of claim 72, wherein the cancer cell is ex vivo or in vivo.

77. The method of claim 72, including determining whether a pattern of expression levels, at different points in time, exists, thereby allowing monitoring of development of a cancer in a subject.

78. A method of identifying a cancer patient less likely to respond to a cardiotonic steroid (CTS) therapy and thus requires a combination of a CTS and a mTOR-inhibitor for treatment the method comprising:
determining Na/K-ATPase levels or activity in a biological sample from the cancer patient,
wherein a change in Na/K-ATPase levels or activity, as compared to Na/K-ATPase levels or activity found in a normal healthy subject, indicates the patient is less likely to respond to CTS therapy.

79. The method of claim 78, wherein the cancer cell is a breast cancer cell.

80. The method of claim 78, wherein the cancer cell is a prostate cancer cell.

81. The method of claim 78, wherein the cancer cell is ex vivo or in vivo.

82. The method of claim 78, including determining whether a pattern of expression levels, at different points in time, exists, thereby allowing monitoring of development of a cancer in a subject.

83. A method of diagnosing a cancer and/or providing a prognosis for patient having a cancer that has altered expression of molecular signaling pathways triggered by ouabain, the method comprising:
contacting a cell from the patient with an antibody that specifically binds to protein that is part of a molecular signaling pathway triggered by ouabain, wherein the molecular signaling pathway is a functional or activated PI3-K/Akt/mTOR pathway; and
determining whether or not expression of the protein is altered in the sample, thereby diagnosing or providing the prognosis for the cancer.

84. The method of claim 83, wherein a therapy resistant cancer has an altered expression of the molecular signaling pathway triggered by ouabain.

85. The method of claim 83, wherein the cell is a breast cancer cell.

86. The method of claim 83, wherein the cell is a prostate cancer cell.

87. The method of claim 83, wherein the cell is ex vivo or in vivo.
